SEQUENCE LISTING (2 sequences)

SEQ ID NO: 1 - DNA, 27 bp, Artificial sequence
  Synthetic
ccttagagtacaaacggaacacgagaa

SEQ ID NO: 2 - DNA, 41 bp, Artificial sequence
  Synthetic
ttctcgtgttccgtttgtactctaaggtggatttttttttt

(19) United States
(12) Patent Application Publication
(10) Pub. No.: US 2019/0376963 A1
(43) Pub. Date: Dec. 12, 2019

(54) SEQUENTIAL SAMPLING METHOD FOR IMPROVING IMMUNOASSAY SENSITIVITY AND KINETICS OF SMALL VOLUME SAMPLES

(71) Applicant: Abbott Laboratories, Abbott Park, IL (US)

(72) Inventors: Sergey Tetin, Abbott Park, IL (US); Jeffrey B. Huff, Abbott Park, IL (US); Joseph P. Skinner, Abbott Park, IL (US); Patrick MacDonald, Abbott Park, IL (US); Qiaoqiao Ruan, Abbott Park, IL (US)

(21) Appl. No.: 16/403,165

(22) Filed: May 3, 2019

Related U.S. Application Data
(60) Provisional application No. 62/667,238, filed on May 4, 2018.

(51) Int. Cl. G01N 33/543; G01N 33/58

(52) U.S. Cl. CPC G01N 33/54393; G01N 33/581; G01N 33/582

(57) ABSTRACT

The disclosure provides a method for an enhanced detection of an analyte present in a biological sample. After the formation of the analyte/specific binding member(s)/detectable label complex, the labels are eluted and a first aliquot of eluant is brought into contact with a solid support, wherein the solid support comprises immobilized thereto specific binding member that specifically binds to the label, removing the first aliquot from the solid support and contacting the solid support with a second aliquot of the eluted label, and repeating the above steps, such that the label is concentrated on the solid support for further analysis to quantify the analyte in the biological sample.

### CROSS REFERENCE TO RELATED APPLICATIONS

This application claims the benefit of U.S. Provisional Patent Application No. 62/667,238, filed May 4, 2018, the disclosure of which is incorporated by reference herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 726 Byte ASCII (Text) file named “36422-US-2-ORD ST25.TXT,” created on May 3, 2019.

### BACKGROUND OF THE INVENTION

Methods and devices that can accurately analyze analytes of interest in a sample are essential for diagnostics, prognostics, environmental assessment, food safety, detection of chemical or biological warfare agents, and the like. Such methods and devices need to be accurate, precise, and sensitive. It is also advantageous if very small sample volumes can be analyzed quickly with minimal instrumentation. While newer detection technologies, such as single molecule counting can detect very small amounts of analyte in a sample, such methods often produce variable results due to loading and sampling errors. As such, there is a need for methods and devices with improved sample analysis capabilities of small volumes.

### BRIEF SUMMARY OF THE INVENTION

The disclosure provides a method for detecting an analyte present in a biological sample. The method comprises (a) providing a volume of a biological sample suspected of containing an analyte; (b) contacting a solid support with first aliquot of the volume of the biological sample, wherein the solid support comprises a first specific binding member that specifically binds to the analyte immobilized thereto; (c) contacting the solid support/first specific binding member/analyte complex with a second specific binding member that specifically binds to the analyte and comprises a detachable detectable label attached thereto, wherein a solid support/first specific binding member/analyte/second specific binding member complex is formed; (d) separating and eluting the detectable label from complex bound to the solid support; (e) transferring an aliquot of detectable label to a second solid support comprising a third specific binding member that specifically binds the detectable label; (f) removing the first aliquot from the solid support and contacting the solid support with a second aliquot of the eluted detectable label; (g) repeating steps (e) and (f) 5 to 30 times, wherein a solid support/third specific binding member/detectable label complex is formed; (h) removing any detectable label not bound to the solid support; and (g) quantifying the analyte by assessing a signal produced by the detectable label.

### BRIEF DESCRIPTION OF THE DRAWING(S)

FIG. 1A is a series of raw TIRF images showing the results of the single molecule counting sensitivity model described in Example 1. FIG. 1B is a graph which illustrates the median number of fluorescent peaks/frame measured with SM-TIRF and a peak finding algorithm. The insert of FIG. 1B is an expansion of the low concentration range. Error bars represent the standard deviation across three independent experiments.

FIG. 2 is a graph illustrating the results of the microparticle assay with SM detection described in Example 2. The graph plots the number of peaks/frame versus the initial, unconcentrated “analyte” concentrations, while the insert shows the low concentration range (Error bars: standard deviation, n=3).

FIG. 3A is a diagram illustrating the procedure for removal of the aliquot from the solid support by pumping of air. FIG. 3B is a graph illustrating the results of analyte concentration using the repeat sampling method described in Example 3. The initial background sample shows the results of measurement prior to adding any conjugate, while the second saturation sample underwent a 60-minute incubation with the conjugate. The remaining samples are a series of aliquots from one stock solution which have been loaded and reloaded into the same well. Each incubation period was 2 minutes, and the well was washed before each measurement. The background level has been colored white across all samples, and the right axis shows the re-zeroed peak counts.

FIG. 4A is a graph illustrating the results of sample reloading from the respective stocks described in Example 3 for each sample with SM-TIRF measurements taken after the initial, 10th, 30th, and 50th reloads. FIG. 4B is a graph which plots the data in FIG. 4A against the stock concentration to demonstrate that the relative relationship between samples is maintained throughout the reloading concentration procedure. The error bars display the standard deviation for the 40 image acquisitions within a given sample measurement.

FIGS. 5A and 5B are graphs illustrating the results of the HIV p24 microparticle assay with single molecule detection described in Example 4. FIG. 5A shows the results for the initial load of eight concentrations of p24 antigen calibrator. The number of SM-TIRF detected peaks from a single 2-minute incubation of each eluted sample is plotted against the initial calibrator concentration. FIG. 5B shows the results following loading of nine more aliquots (total=10) from the eluted samples. The SM peaks are plotted against the same initial p24 concentrations, and a boost in total peaks and a reduction in relative error was observed. SM counting achieved a sensitivity of ˜80 fM in a standard immunoassay application (Error bars: standard deviation, frames=40).

FIG. 6 is a table detailing the input parameters for the experiments described in Example 5.

FIGS. 7A-7C are plots of real-time antigen binding curves for the three different sample loading and incubation conditions described in Example 5: 1×1.1 μl for 5 minutes (FIG. 7A), 5.5 μl for 5 minutes (FIG. 7B), and 5×1.1 μl for 1 minute each (FIG. 7C).

### DETAILED DESCRIPTION OF THE INVENTION

The present disclosure is predicated, at least in part, on the discovery that a sample reloading approach for immunoassay of small volume samples can be used to concentrate the sample on a detection surface for the purposes of single molecule detection. This repeated sampling approach provides for maximum analyte capture, thus leading to improved sensitivity, and a minimum amount of variability in interrogating a given sample, thus leading to an improved coefficient of variation as compared to methods that do not employ repeat sampling.

The disclosure provides a method for detecting an analyte present in a biological sample. The method may involve single molecule detection and counting. In certain embodiments, the disclosed method may be used for determining the presence and/or concentration of one or more analytes in a sample.

### Biological Sample

As used herein, the terms “biological sample,” “sample,” and “test sample” are used interchangeably and refer to a substance containing or suspected of containing an analyte of interest. The biological sample may be derived from any suitable source. For example, the source of the biological sample may be synthetic (e.g., produced in a laboratory), or a naturally-occurring substance obtained or derived from, e.g., the environment (e.g., air, soil, fluid samples, e.g., water supplies, etc.), an animal (e.g., a mammal), a plant, or another organism. In one embodiment, the source of the biological sample is a human bodily substance (e.g., bodily fluid, blood, serum, plasma, urine, saliva, sweat, sputum, semen, mucus, lacrimal fluid, lymph fluid, amniotic fluid, interstitial fluid, lung lavage, cerebrospinal fluid, feces, tissue, an organ, and the like). Human tissues may include, but are not limited to, skeletal muscle tissue, liver tissue, lung tissue, kidney tissue, myocardial tissue, brain tissue, bone marrow, cervix tissue, skin, and the like. In some embodiments, the source of the sample may be a biopsy sample, which may be solubilized by tissue disintegration/cell lysis. The sample may be a liquid sample, a liquid extract of a solid sample, a fluent particulate solid, or fluid suspension of solid particles.

The disclosed method involves providing a volume of a biological sample suspected of containing an analyte. Any suitable volume of the sample may be provided. It will be appreciated that single molecule (SM) detection methods typically involve small sample volumes. In this regard, the volume of the biological sample may be about 10 μl to about 50 μl (e.g., 10 μl, 15 μl, 20 μl, 25 μl, 30 μl, 35 μl, 40 μl, or 50 μl). In another embodiment, the volume of the biological sample may be about 10 μl to about 30 μl (e.g., 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, or a range defined by any two of the foregoing values).

The disclosed method comprises contacting a solid support with first, second, and subsequent aliquots of the volume of biological sample. The term “aliquot,” as used herein, refers to a portion of a total amount or volume of a liquid. In the context of the disclosure, each of the first, second, and subsequent aliquots may be of any suitable volume. In one embodiment, each of the first, second, and subsequent aliquots comprises about 1 nl to about 2 μl of the volume of the biological sample (e.g., 1 nl, 10 nl, 50 nl, 100 nl, 200 nl, nl, 300 nl, 400 nl, 500 nl, 600 nl, 700 nl, 800 nl, 900 nl, 1 μl, 1.5 μl, 2 or a range defined by any two of the foregoing values). For example, an aliquot may comprise about 500 nl to about 1 μl (e.g., 525 nl, 550 nl, 575 nl, 625 nl, 650 nl, 675 nl, 725 nl, 750 nl, 775 nl, 825 nl, 850 nl, 875 nl, 925 nl, 950 nl, or 975 nl) or about 1 μl to about 2 μl (e.g., 1.1 μl, 1.2 μl, 1.3 μl, 1.4 μl, 1.5 μl, 1.6 μl, 1.7 μl, 1.8 μl or 1.9 μl) of the volume of the biological sample. In one embodiment, each of the first, second, and subsequent aliquots comprises about 1 μl of the volume of the biological sample.

In some embodiments, a liquid biological sample may be diluted prior to use in an assay. For example, in embodiments where the biological sample is a human body fluid (e.g., blood or serum), the fluid may be diluted with an appropriate solvent (e.g., PBS buffer). A fluid sample may be diluted about 1-fold, about 2-fold, about 3-fold, about 4-fold, about 5-fold, about 6-fold, about 10-fold, about 100-fold, or greater, prior to use.

In other embodiments, the sample may undergo pre-analytical processing. Pre-analytical processing may offer additional functionality, such as nonspecific protein removal and/or effective yet inexpensive implementable mixing functionality. General methods of pre-analytical processing include, for example, the use of electrokinetic trapping, AC electrokinetics, surface acoustic waves, isotachophoresis, dielectrophoresis, electrophoresis, and other pre-concentration techniques known in the art. In some cases, a liquid sample may be concentrated prior to use in an assay. For example, in embodiments where biological sample is a human body fluid (e.g., blood, serum), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

### Analytes

The terms “analyte,” “target analyte,” and “analyte of interest” are used interchangeably herein and refer to the substance being measured in the disclosed method. As will be appreciated by those in the art, any analyte that can be specifically bound by a first specific binding member and a second specific binding member may be detected, and optionally quantified, using the methods of the present disclosure.

In some embodiments, the analyte may be a biomolecule. Examples of suitable biomolecules include, but are not limited to, macromolecules such as, proteins, lipids, and carbohydrates. Other biomolecules include, for example, hormones, antibodies, growth factors, oligonucleotides, polynucleotides, haptens, cytokines, enzymes, receptors (e.g., neural, hormonal, nutrient, and cell surface receptors) or their ligands, cancer markers (e.g., PSA, TNF-alpha), markers of myocardial infarction (e.g., BNP, troponin, creatine kinase, and the like), toxins, metabolic agents (e.g., vitamins), and the like. Suitable protein analytes include, for example, peptides, polypeptides, protein fragments, protein complexes, fusion proteins, recombinant proteins, phosphoproteins, glycoproteins, lipoproteins, and the like.

In certain embodiments, the analyte may be a post-translationally modified protein (e.g., phosphorylated, methylated, glycosylated protein) and the first or the second specific binding member may be an antibody specific to the post-translational modification. A modified protein may be bound to a first specific binding member immobilized on a solid support where the first specific binding member binds to the modified protein but not the unmodified protein. In other embodiments, the first specific binding member may bind to both the unmodified and the modified protein, and the second specific binding member may be specific to the post-translationally modified protein.

A non-limiting list of analytes that may be analyzed by the methods disclosed herein include Aβ342 amyloid beta-protein, fetuin-A, tau, secretogranin II, prion protein, alpha-synuclein, tau protein, NSE, S100B, NF-L, ApoA1, BDNF, MBP, Sodium creatinine, BUN, AMPAR,_prion protein, neurofilament light chain, parkin, PTEN induced putative kinase 1, DJ-1, leucine-rich repeat kinase 2, mutated ATP13A2, Apo H, ceruloplasmin, peroxisome proliferator-activated receptor gamma coactivator-1 alpha (PGC-1α), transthyretin, vitamin D-binding protein, proapoptotic kinase R (PKR) and its phosphorylated PKR (pPKR), CXCL13, IL-12p40, CXCL13, IL-8, Dkk-3 (semen), p14 endocan fragment, Serum, ACE2, autoantibody to CD25, hTERT, CAI25 (MUC 16), VEGF, sIL-2, osteopontin, human epididymis protein 4 (HE4), alpha-fetoprotein, albumin, albuminuria, microalbuminuria, neutrophil gelatinase-associated lipocalin (NGAL), interleukin 18 (IL-18), kidney injury molecule-1 (KIM-1), liver fatty acid binding protein (L-FABP), LMP1, BARF1, IL-8, carcinoembryonic antigen (CEA), BRAF, CCNI, EGRF, FGF19, FRS2, GREB1, LZTS1, alpha-amylase, carcinoembryonic antigen, CA 125, IL8, thioredoxin, beta-2 microglobulin, tumor necrosis factor-alpha receptors, CA15-3, follicle-stimulating hormone (FSH), leutinizing hormone (LH), T-cell lymphoma invasion and metastasis 1 (TIAM1), N-cadherin, EC39, amphiregulin, dUTPase, secretory gelsolin (pGSN), prostate specific antigen (PSA), thymosin 015, insulin, plasma C-peptide, glycosylated hemoglobin (HBA1c), C-Reactive Protein (CRP), Interleukin-6 (IL-6), Rho GDP-dissociation inhibitor 2 (ARHGDIB), cofilin-1 (CFL1), profilin-1 (PFN1), glutathione S-transferase P (GSTP1), protein S100-A11 (S100A11), peroxiredoxin-6 (PRDX6), 10 kDa heat shock protein, mitochondrial (HSPE1), lysozyme C precursor (LYZ), glucose-6-phosphate isomerase (GPI), histone H2A type 2-A (HIST2H2AA), glyceraldehyde-3-phosphate dehydrogenase(GAPDH), basement membrane-specific heparin sulfate proteoglycan core protein precursor (HSPG2), galectin-3-binding protein precursor (LGALS3BP), cathepsin D precursor (CTSD), apolipoprotein E precursor (APOE), Ras GTPase-activating-like protein (IQGAP1), ceruloplasmin precursor (CP), and IGLC2, PCDGF/GP88, EGFR, HER2, MUC4, IGF-IR, p27(kipl), Akt, HER3, HER4, PTEN, PIK3CA, SHIP, Grb2, Gab2, 3-phosphoinositide dependent protein kinase-1 (PDK-1), TSC1, TSC2, mTOR, ERBB receptor feedback inhibitor 1 (MIG-6), S6K, src, KRAS, mitogen-activated protein kinase 1 (MEK), cMYC, topoisomerase (DNA) II alpha 170 kDa, FRAP1, NRG1, ESR1, ESR2, PGR, CDKN1B, MAP2K1, NEDD4-1, FOXO3A, PPP1R1B, PXN, ELA2, CTNNB1, AR, EPHB2, KLF6, ANXA7, NKX3-1, PITX2, MKI67, PHLPP, adiponectin (ADIPOQ), fibrinogen alpha chain (FGA), leptin (LEP), advanced glycosylation end product-specific receptor (AGER or RAGE), alpha-2-HS-glycoprotein (AHSG), angiogenin (ANG), CD14, ferritin (FTH1), insulin-like growth factor binding protein 1 (IGFBP1), interleukin 2 receptor, alpha (IL2RA), vascular cell adhesion molecule 1 (VCAM1), Von Willebrand factor (VWF), myeloperoxidase (MPO), IL1α, TNFα, perinuclear anti-neutrophil cytoplasmic antibody (p-ANCA), lactoferrin, calprotectin, Wilm's Tumor-1 protein, Aquaporin-1, MLL3, AMBP, VDAC1, E. coli enterotoxins (heat-labile exotoxin, heat-stable enterotoxin), influenza HA antigen, tetanus toxin, diphtheria toxin, botulinum toxins, Shiga toxin, Shiga-like toxin I, Shiga-like toxin II, Clostridium difficile toxins A and B, drugs of abuse (e.g., cocaine), protein biomarkers (including, but not limited to, nucleolin, nuclear factor-kB essential modulator (NEMO), CD-30, protein tyrosine kinase 7 (PTK7), MUC1 glycoform, immunoglobulin μ heavy chains (IGHM), immunoglobulin E, αvβ3 integrin, α-thrombin, HIV gp120, HIV p24, NF-κB, E2F transcription factor, plasminogen activator inhibitor, Tenascin C, CXCL12/SDF-1, and prostate specific membrane antigen (PSMA).

The analyte may be a cell, such as, for example, gastric cancer cells (e.g., HGC-27 cells); non-small cell lung cancer (NSCLC) cells, colorectal cancer cells (e.g., DLD-1 cells), H23 lung adenocarcinoma cells, Ramos cells, T-cell acute lymphoblastic leukemia (T-ALL) cells, CCRF-CEM cells, acute myeloid leukemia (AML) cells (e.g., HL60 cells), small-cell lung cancer (SCLC) cells (e.g., NCI-H69 cells), human glioblastoma cells (e.g., U118-MG cells), prostate cancer cells (e.g., PC-3 cells), HER-2-overexpressing human breast cancer cells (e.g., SK-BR-3 cells), pancreatic cancer cells (e.g., Mia-PaCa-2)). In other embodiments, the analyte may be an infectious agent, such as a bacterium (e.g., Mycobacterium tuberculosis, Staphylococcus aureus, Shigella dysenteriae, Escherichia coli O157:H7, Campylobacter jejuni, Listeria monocytogenes, Pseudomonas aeruginosa, Salmonella 08, and Salmonella enteritidis), virus (e.g., retroviruses (such as HIV), herpesviruses, adenoviruses, lentiviruses, Filoviruses (e.g., West Nile, Ebola, and Zika viruses), hepatitis viruses (e.g., A, B, C, D, and E); HPV, Parvovirus, etc.), a parasite, or fungal spores.

### Specific Binding Members

The disclosed method comprises contacting a solid support with a first aliquot of the volume of the biological sample, wherein the solid support comprises immobilized thereto a first specific binding member that specifically binds to the analyte. The terms “specific binding partner” and “specific binding member” are used interchangeably herein and refer to one of two or more different molecules that specifically recognize the other molecule compared to substantially less recognition of other molecules. The one of two different molecules has an area on the surface or in a cavity, which specifically binds to and is thereby defined as complementary with a particular spatial and polar organization of the other molecule. The molecules may be members of a specific binding pair. For example, a specific binding member may include, but is not limited to, a protein, such as a receptor, an enzyme, and an antibody.

It will be appreciated that the choice of binding members (e.g., first, second, third, fourth, or subsequent binding members) will depend on the analyte or analytes to be analyzed. Binding members for a wide variety of target molecules are known or can be readily found or developed using known techniques. For example, when the target analyte is a protein, the binding members may include peptides, proteins, particularly antibodies or fragments thereof (e.g., antigen-binding fragments (Fabs), Fab′ fragments, and F(ab′)₂ fragments), full-length monoclonal or polyclonal antibodies, antibody-like fragments, recombinant antibodies, chimeric antibodies, single-chain Fvs (“scFv”), single chain antibodies, single domain antibodies, such as variable heavy chain domains (“VHH”; also known as “VHH fragments”) derived from animals in the Camelidae family (see, e.g., Gottlin et al., Journal of Biomolecular Screening, 14:77-85 (2009)), recombinant VHH single-domain antibodies, VNAR fragments, disulfide-linked Fvs (“sdFv”), anti-idiotypic (“anti-Id”) antibodies, and functionally active epitope-binding fragments of any of the foregoing. The binding members also can be other proteins, such as receptor proteins, Protein A, Protein C, or the like. When the analyte is a small molecule, such as a steroid, bilin, retinoid, or lipid, the first and/or the second specific binding member may be a scaffold protein (e.g., lipocalins) or a receptor. In some embodiments, a specific binding member for protein analytes can be a peptide. In another embodiment, when the target analyte is an enzyme, suitable binding members may include enzyme substrates and/or enzyme inhibitors, such as a peptide, a small molecule, and the like. In some cases, when the target analyte is a phosphorylated species, the binding members may comprise a phosphate-binding agent. For example, the phosphate-binding agent may comprise metal-ion affinity media such as those described in U.S. Pat. No. 7,070,921 and U.S. Patent Application Publication 2006/0121544.

When the analyte is a carbohydrate, potentially suitable specific binding members (as defined herein) include, for example, antibodies, lectins, and selectins. As will be appreciated by those of ordinary skill in the art, any molecule that can specifically associate with a target analyte of interest may potentially be used as a binding member.

In certain embodiments, suitable target analyte/binding member complexes can include, but are not limited to, antibodies/antigens, antigens/antibodies, receptors/ligands, ligands/receptors, proteins/nucleic acid, enzymes/substrates and/or inhibitors, carbohydrates (including glycoproteins and glycolipids)/lectins and/or selectins, proteins/proteins, proteins/small molecules, etc.

Certain embodiments utilize binding members that are proteins or polypeptides. As is known in the art, any number of techniques may be used to attach a polypeptide to a solid support. A wide variety of techniques are known for adding reactive moieties to proteins, such as, for example, the method described in U.S. Pat. No. 5,620,850. Methods for attachment of proteins to surfaces also are described in, for example, Heller, Acc. Chem. Res., 23: 128 (1990).

As described herein, binding between the specific binding members and the analyte is specific, e.g., as when the binding member and the analyte are complementary parts of a binding pair. For example, in one embodiment, the binding member may be an antibody that binds specifically to an epitope on an analyte. The antibody, according to one embodiment, can be any antibody capable of binding specifically to an analyte of interest. For example, appropriate antibodies include, but are not limited to, monoclonal antibodies, bispecific antibodies, minibodies, domain antibodies (dAbs) (e.g., such as described in Holt et al., Trends in Biotechnology, 21: 484-490 (2014)), single domain antibodies (sdAbs) that are naturally occurring, e.g., as in cartilaginous fishes and camelid, or which are synthetic, e.g., nanobodies, VHH, or other domain structure), synthetic antibodies (sometimes referred to as antibody mimetics), chimeric antibodies, humanized antibodies, antibody fusions (sometimes referred to as “antibody conjugates”), and fragments thereof. As another example, the analyte molecule may be an antibody, the first specific binding member may be an antigen, and the second specific binding member may be a secondary antibody that specifically binds to the target antibody. Alternatively, the first specific binding member may be a secondary antibody that specifically binds to the target antibody and the second specific binding member may be an antigen. In other embodiment, the analyte molecule may be an antibody and the binding member may be a peptide that binds specifically to the antibody.

In some embodiments, the first or second specific binding member may be a chemically programmed antibody (cpAb) (Rader, Trends in Biotechnology, 32:186-197 (2014)), bispecific cpAbs, antibody-recruiting molecules (ARMs) (McEnaney et al., ACS Chem. Biol., 7: 1139-1151 (2012)), branched capture agents, such as a triligand capture agent (Millward et al., J. Am. Chem. Soc., 133: 18280-18288 (2011)), engineered binding proteins derived from non-antibody scaffolds, such as monobodies (derived from the tenth fibronectin type III domain of human fibronectin), affibodies (derived from the immunoglobulin binding protein A), DARPins (based on Ankyrin repeat modules), anticalins (derived from the lipocalins bilin-binding protein and human lipocalin 2), and cysteine knot peptides (knottins) (Gilbreth and Koide, Current Opinion in Structural Biology, 22:1-8 (2012); Banta et al., Annu. Rev. Biomed. Eng., 15: 93-113 (2013)), WW domains (Patel et al., Protein Engineering, Design & Selection, 26(4): 307-314 (2013)), repurposed receptor ligands, affitins (Béhar et al., Protein Engineering, Design & Selection, 26: 267-275 (2013)), and/or Adhirons (Tiede et al., Protein Engineering, Design & Selection, 27: 145-155 (2014)).

In embodiments where the analyte is a cell (e.g., mammalian, avian, reptilian, other vertebrate, insect, yeast, bacterial, cell, etc.), the specific binding members may be ligands having specific affinity for a cell surface antigen (e.g., a cell surface receptor). In one embodiment, the specific binding member may be an adhesion molecule receptor or portion thereof, which has binding specificity for a cell adhesion molecule expressed on the surface of a target cell type. The adhesion molecule receptor binds with an adhesion molecule on the extracellular surface of the target cell, thereby immobilizing or capturing the cell. The bound cell may then be detected by using a second binding member that may be the same as the first binding member or may bind to a different molecule expressed on the surface of the cell.

In some embodiments, the binding affinity between analyte molecules and specific binding members should be sufficient to remain bound under the conditions of the assay, including wash steps to remove molecules or particles that are non-specifically bound. In some embodiments, for example, in the detection of certain biomolecules, the binding constant of the analyte molecule to its complementary binding member may be between at least about 10⁴ and about 10⁶ M⁻¹, at least about 10⁵ and about 10⁹ M⁻¹, at least about 10⁷ and about 10⁹ M⁻¹, greater than about 10⁹ M⁻¹.

The solid support having a surface on which a first specific binding member is immobilized may be any suitable surface in planar or non-planar conformation, such as, for example, a surface of a microfluidic chip, an interior surface of a chamber, a bead, an exterior surface of a bead, an interior and/or exterior surface of a porous bead, a particle, a microparticle, an electrode, a slide (e.g., a glass slide), or a multiwell (e.g., a 96-well) plate. In one embodiment, the first specific binding member may be attached covalently or non-covalently to a bead, e.g., latex, agarose, sepharose, streptavidin, tosylactivated, epoxy, polystyrene, amino bead, amine bead, carboxyl bead, and the like. In certain embodiments, the bead may be a particle, e.g., a microparticle (MP). In some embodiments, the microparticle may be between about 0.1 nm and about 10 microns, between about 50 nm and about 5 microns, between about 100 nm and about 1 micron, between about 0.1 nm and about 700 nm, between about 500 nm and about 10 microns, between about 500 nm and about 5 microns, between about 500 nm and about 3 microns, between about 100 nm and 700 nm, or between about 500 nm and 700 nm. For example, the microparticle may be about 4-6 microns, about 2-3 microns, or about 0.5-1.5 microns. Particles less than about 500 nm are sometimes considered nanoparticles. Thus, the microparticle optionally may be a nanoparticle between about 0.1 nm and about 500 nm, between about 10 nm and about 500 nm, between about 50 nm and about 500 nm, between about 100 nm and about 500 nm, about 100 nm, about 150 nm, about 200 nm, about 250 nm, about 300 nm, about 350 nm, about 400 nm, about 450 nm, or about 500 nm.

In other embodiments, the bead may be a magnetic bead or a magnetic particle.

Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, NiO/Fe. Examples of ferrimagnetic materials include NiFe₂O₄, CoFe₂O₄, Fe₃O₄ (or FeO.Fe₂O₃). Beads can have a solid core portion that is magnetic and is surrounded by one or more non-magnetic layers. Alternatively, the magnetic portion can be a layer around a non-magnetic core. The solid support on which the first specific binding member is immobilized may be stored in dry form or in a liquid. The magnetic beads may be subjected to a magnetic field prior to or after contacting with the sample with a magnetic bead on which the first specific binding member is immobilized.

A specific binding member may be attached to the solid support using any suitable method, a variety of which are known in the art. For example, a specific binding member may be attached to a solid support via a linkage, which may comprise any moiety, functionalization, or modification of the support and/or binding member that facilitates the attachment of the binding member to the support. The linkage between the binding member and the support may include one or more chemical or physical bonds and/or chemical spacers providing such bond(s) (e.g., non-specific attachment via van der Waals forces, hydrogen bonding, electrostatic interactions, hydrophobic/hydrophilic interactions; etc.). Any number of techniques may be used to attach a polypeptide to a wide variety of solid supports, such as those described in, for example U.S. Pat. No. 5,620,850, and Heller, Acc. Chem. Res., 23: 128 (1990).

In certain embodiments, a solid support may also comprise a protective, blocking, or passivating layer that can eliminate or minimize non-specific attachment of non-capture components (e.g., analyte molecules, binding members) to the binding surface during the assay which may lead to false positive signals during detection or loss of signal. Examples of materials that may be utilized in certain embodiments to form passivating layers include, but are not limited to, polymers, such as poly(ethylene glycol), that repel the non-specific binding of proteins; naturally occurring proteins with this property, such as serum albumin and casein; surfactants, e.g., zwitterionic surfactants, such as sulfobetaines; naturally occurring long-chain lipids; polymer brushes, and nucleic acids, such as salmon sperm DNA.

The solid support may be contacted with a first aliquot of the volume of the sample using any suitable method known in the art. The term “contacting,” as used herein, refers to any type of combining action which brings a binding member into sufficiently close proximity with an analyte of interest in a sample such that a binding interaction will occur if the analyte of interest specific for the binding member is present in the sample. Contacting may be achieved in a variety of different ways, including combining the sample with a binding member, exposing a target analyte to a binding member by introducing the binding member in close proximity to the analyte, and the like. The contacting may be repeated as many times as necessary.

Whatever method is used, the solid support is contacted with the first aliquot of the volume of sample under conditions whereby any analyte present in the first aliquot binds to the first specific binding member immobilized on the solid support. In one embodiment, contact between the solid support and first aliquot is maintained (i.e., incubated) for a sufficient period of time to allow for the binding interaction between the first specific binding member and analyte to occur. In one embodiment, the first aliquot is incubated on the solid support for at least 30 seconds and at most 10 minutes. For example, the first aliquot may be incubated with the solid support for about 1, 2, 3, 4, 5, 6, 7, 8, or 9 minutes. In one embodiment, the first aliquot may be incubated with the solid support for about 2 minutes. In addition, the incubating may be in a binding buffer that facilitates the specific binding interaction, such as, for example, albumin (e.g., BSA), non-ionic detergents (Tween-20, Triton X-100), and/or protease inhibitors (e.g., PMSF). The binding affinity and/or specificity of a specific binding member may be manipulated or altered in the assay by varying the binding buffer. In some embodiments, the binding affinity and/or specificity may be increased by varying the binding buffer. In some embodiments, the binding affinity and/or specificity may be decreased by varying the binding buffer. Other conditions for the binding interaction, such as, for example, temperature and salt concentration, may also be determined empirically or may be based on manufacturer's instructions. For example, the contacting may be carried out at room temperature (21° C.-28° C., e.g., 23° C.-25° C.), 37° C., or 4° C.

Following a sufficient incubation time between the solid support and first aliquot of the volume of the biological sample to allow an analyte in the aliquot to bind the first specific binding member, the disclosed method comprises removing the first aliquot from the solid support and contacting the sold support with a second aliquot of the biological sample. The first aliquot may be removed from the solid support using any suitable method, such as, for example, introducing an amount of air onto the solid support (e.g., a well) such that the force of the air displaces the first aliquot from the solid support. Alternatively, the first aliquot may be removed by introducing the second (or subsequent) aliquots onto the solid support, such that first aliquot is displaced from the solid support. Embodiments relating to the first aliquot described herein also are applicable to the same aspects of the second aliquot (and subsequent aliquots as described below).

The disclosed method further comprises repeating the steps of (i) contacting a solid support with an aliquot of the volume of the biological sample; and (ii) removing the aliquot from the solid support and contacting the solid support with a second aliquot of the volume of the biological sample such that a solid support/first specific binding member/analyte complex is formed. In other words, the solid support is contacted with a first, second, and subsequent aliquots of the volume of the biological sample, and each aliquot is removed from the solid support prior to application of the next subsequent aliquot to the solid support. In this manner, an analyte of interest may be concentrated on the solid support in the form of a solid support/first specific binding member/analyte complex and detected as described further herein. As used herein, the term “complex” refers to at least two molecules that are specifically bound to one another. Examples of complexes include, but are not limited to, an analyte bound to an analyte-binding molecule (e.g., an antibody), an analyte bound to a plurality of analyte-binding molecules, e.g., an analyte bound to two analyte-binding molecules, an analyte-binding molecule bound to a plurality of analytes, e.g., an analyte-binding molecule bound to two analytes.

It is believed that the “repeat sampling” method described herein provides for capture and concentration of the maximum amount of analyte, leading to improved immunoassay sensitivity, while producing a minimum amount of variability in interrogating a given sample, resulting in an improved coefficient of variation (CV). The present disclosure, in particular, demonstrates that the disclosed “repeat sampling” method enhances the sensitivity of single molecule detection systems, such as those described herein and known in the art (e.g., total internal reflection fluorescence (TIRF) microscopy). Furthermore, the repeat sampling method allows one of ordinary skill in the art to take advantage of a re-distribution of analyte equilibrium with each addition of fresh aliquot of the biological sample volume.

The steps of contacting the solid support with an aliquot of the volume of the biological sample, removing the aliquot from the solid support, and contacting the solid support with a second (or subsequent) aliquot of the volume of the biological sample may be repeated any number of times to allow for sufficient formation of a solid support/first specific binding member/analyte complex. In this regard, the steps may be repeated at least 5 times and not more than 30 times (e.g., 5, 10, 15, 20, 25, or 30 times). For example, the steps may repeated 10 to 20 times (e.g., 10, 11, 12, 13, 14, 15, 16, 17, 18, 19, or 20 times) or 20 to 30 times (e.g., 21, 22, 23, 24, 25, 26, 27, 28, 29, or 30 times). In one embodiment, the contacting and removal steps are repeated 10 times.

After the contacting and removal steps are sufficiently repeated to form a solid support/first specific binding member/analyte complex and concentrate the complex on the solid support, the method comprises contacting the solid support/first specific binding member/analyte complex with a second specific binding member that specifically binds to the analyte and comprises a detectable label attached thereto, wherein a solid support/first specific binding member/analyte/second specific binding member complex is formed.

As discussed above with respect to contacting the solid support with the first, second, and subsequent aliquots of the biological sample, contacting the solid support/first specific binding member/analyte complex with a second specific binding member may be carried out under conditions sufficient for a binding interaction between the analyte and the second binding member to occur. Following this contacting step, any second specific binding member not bound to the analyte may be removed, followed by an optional wash step. Any unbound second specific binding member may be separated from the complex of the solid support/first specific binding member/analyte/second specific binding member by any suitable means such as, for example, droplet actuation, electrophoresis, electrowetting, dielectrophoresis, electrostatic actuation, electric field mediated, electrode mediated, capillary force, chromatography, centrifugation, aspiration, or surface acoustic wave (SAW)-based washing methods.

The disclosed method may comprise quality control components. “Quality control components” in the context of immunoassays and kits described herein, include, but are not limited to, calibrators, controls, and sensitivity panels. A “calibrator” or “standard” can be used (e.g., one or more, such as a plurality) in order to establish calibration (standard) curves for interpolation of the concentration of an analyte, such as an antibody. Alternatively, a single calibrator, which is near a reference level or control level (e.g., “low”, “medium”, or “high” levels), can be used. Multiple calibrators (i.e., more than one calibrator or a varying amount of calibrator(s)) can be used in conjunction to comprise a “sensitivity panel.” The calibrator is optionally, and is preferably, part of a series of calibrators in which each of the calibrators differs from the other calibrators in the series, such as, for example, by concentration or detection method (e.g., colorimetric or fluorescent detection).

The repeated sampling technique described herein may also comprise an elution step that may also be repeated, which serves to further enrich the analyte for detection. For example, following formation of the solid support/first specific binding member/analyte/second specific binding member complex, a first aliquot of the complex may be eluted and placed onto a detection surface (e.g., a microfluidic channel on a detection slide) coated with streptavidin. Analyte molecules conjugated to a detectable label and biotin are then captured by the streptavidin surface, depleting labeled analyte molecules from the complex solution. Following a short incubation (e.g., 1-2 minutes), air may be introduced into the channel of the detection surface so as to displace the “used” aliquot. The bulk of labeled analyte molecules typically are captured in within the first two minutes, while capture of 100% of labeled analyte molecules typically occurs after about 15 minutes. A second “fresh” aliquot of the labeled analyte molecules may be introduced into the channel and incubated for 1-2 minutes, which allows for capture of a new portion of the biotinylated labeled analyte at the streptavidin surface. The channel may be then cleared with air as discussed above, and the process repeated any suitable number of times. In this regard, the elution process may be repeated at least 5 times and not more than 30 times (e.g., 5, 10, 15, 20, 25, or 30 times). For example, the elution process may be repeated 10 to 20 times (e.g., 10, 11, 12, 13, 14, 15, 16, 17, 18, 19, or 20 times) or 20 to 30 times (e.g., 21, 22, 23, 24, 25, 26, 27, 28, 29, or 30 times).

### Analyte Detection and Measurement

As noted above, the second specific binding member comprises a detectable label attached thereto. The terms “label” and “detectable label” are used interchangeably herein and refer to a moiety attached to a specific binding member or analyte to render the reaction between the specific binding member and the analyte detectable, and the specific binding member or analyte so labeled is referred to as “detectably labeled.” A label can produce a signal that is detectable by visual or instrumental means. The detectable label may be, for example, (i) a tag attached to a specific binding member or analyte by a cleavable linker; or (ii) signal-producing substance, such as a chromagen, a fluorescent compound, an enzyme, a chemiluminescent compound, a radioactive compound, and the like. In one embodiment, the detectable label may comprise a moiety that produces light, e.g., an acridinium compound, or a moiety that produces fluorescence, e.g., fluorescein. In another embodiment, the detectable label may comprise one or more nucleic acid molecules capable of producing a detectable signal.

Any suitable signal-producing substance known in the art can be used as a detectable label. For example, the detectable label can be a radioactive label (such as, e.g., ³H, ¹⁴C, ³²P, ³³P, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm), an enzymatic label (such as, e.g., horseradish peroxidase, alkaline peroxidase, glucose 6-phosphate dehydrogenase, and the like, a chemiluminescent label (such as, e.g., acridinium esters, thioesters, sulfonamides, luminol, isoluminol, phenanthridinium esters, and the like), a fluorescent label (such as, e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, rhodamine, phycobiliproteins, and R-phycoerythrin), quantum dots (e.g., zinc sulfide-capped cadmium selenide), a thermometric label, or an immuno-polymerase chain reaction label. A fluorescent label can be used in fluorescence polarization immunoassay (FPIA) (see, e.g., U.S. Pat. Nos. 5,593,896, 5,573,904, 5,496,925, 5,359,093, and 5,352,803). The detectable label may be a molecule that is detectable by electronic means (e.g., a molecule that changes an electrical response, such as current, voltage or resistance). In one embodiment, for example, a molecule passing through a solid-state or biological nanopore can be detected by changing the electrical output of the nanopore.

An acridinium compound can be used as a detectable label in a homogeneous chemiluminescent assay (see, e.g., Adamczyk et al., Bioorg. Med. Chem. Lett., 16: 1324-1328 (2006); Adamczyk et al., Bioorg. Med. Chem. Lett., 4: 2313-2317 (2004); Adamczyk et al., Biorg., Med., Chem., Lett., 14: 3917-3921 (2004); and Adamczyk et al., Org. Lett., 5: 3779-3782 (2003)). In one aspect, the acridinium compound is an acridinium-9-carboxamide. Methods for preparing acridinium 9-carboxamides are described in, for example, Mattingly, J., Biolumin. Chemilumin., 6: 107-114 (1991); Adamczyk et al., J. Org. Chem., 63: 5636-5639 (1998); Adamczyk et al., Tetrahedron, 55: 10899-10914 (1999); Adamczyk et al., Org. Lett., 1: 779-781 (1999); Adamczyk et al., Bioconjugate Chem., 11: 714-724 (2000); Mattingly et al., In: Luminescence Biotechnology: Instruments and Applications; Dyke, K. V. Ed.; CRC Press: Boca Raton, pp. 77-105 (2002); Adamczyk et al., Org. Lett., 5: 3779-3782 (2003); and U.S. Pat. Nos. 5,468,646, 5,543,524 and 5,783,699.

Another example of an acridinium compound is an acridinium-9-carboxylate aryl ester, such as, for example, 10-methyl-9-(phenoxycarbonyl)acridinium fluorosulfonate (available from Cayman Chemical, Ann Arbor, Mich.). Methods for preparing acridinium 9-carboxylate aryl esters are described in, e.g., McCapra et al., Photochem. Photobiol., 4: 1111-21 (1965); Razavi et al., Luminescence, 15: 245-249 (2000); Razavi et al., Luminescence, 15: 239-244 (2000); and U.S. Pat. No. 5,241,070. Such acridinium-9-carboxylate aryl esters are efficient chemiluminescent indicators for hydrogen peroxide produced in the oxidation of an analyte by at least one oxidase in terms of the intensity of the signal and/or the rapidity of the signal.

Detectable labels, labeling procedures, and detection of labels are described in Polak and Van Noorden, Introduction to Immunocytochemistry, 2nd ed., Springer Verlag, N.Y. (1997), and in Haugland, Handbook of Fluorescent Probes and Research Chemicals (1996), Molecular Probes, Inc., Eugene, Oreg.

Upon removal of any unbound second specific binding member from the vicinity of the complex of the solid support/first specific binding member/analyte/second specific binding member, the disclosed method comprises detecting the analyte by assessing a signal produced by the detectable label. The detectable label attached to the second binding member present in the solid support/first specific binding member/analyte/second specific binding member complex may be separated by any suitable means or may be detected using techniques known in the art. Alternatively, in some embodiments, if the detectable label comprises a tag, the tag can be cleaved or disassociated from the complex which remains after removal of unbound reagents. For example, the tag may be attached to the second binding member via a cleavable linker, such as those described in, e.g., International Patent Application Publication WO 2016/161402. The complex of the solid support/first specific binding member/analyte/second specific binding member may be exposed to a cleavage agent that mediates cleavage of the cleavable linker.

Following detection of a signal from the label or tag, the presence or amount of analyte of interest present in a sample can be determined (e.g., quantified) using any suitable method known in the art. Such methods include, but are not limited to, immunoassays. Any suitable immunoassay may be utilized, such as, for example, a sandwich immunoassay (e.g., monoclonal-polyclonal sandwich immunoassays, including enzyme detection (enzyme immunoassay (EIA) or enzyme-linked immunosorbent assay (ELISA)), competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay (e.g., homogeneous chemiluminescent assay), heterogeneous assay, and capture on the fly assay. In some embodiments, one tag is attached to a capture antibody and a detection antibody. Alternately, a microparticle or nanoparticle employed for capture, also can function for detection (e.g., where it is attached or associated by some means to a cleavable linker). Immunoassay components and techniques that may be used in the disclosed method are further described in, e.g., International Patent Application Publication Nos. WO 2016/161402 and WO 2016/161400.

In other embodiments, the methods described herein may be used in conjunction with methodologies for analyzing (e.g., detecting and/or quantifying) an analyte at the single molecule level. Any suitable technique for analyzing single molecules and single molecule interactions may be used in the context of the present disclosure, a variety of which are known in the art. Such single molecule (SM) detection techniques include, but are not limited to, single molecule fluorescence resonance energy transfer (FRET) (see, e.g., Keller et al., J. Am. Chem. Soc., 136: 4534-4543 (2014); and Kobitski et al., Nucleic Acids Res., 35: 2047-2059, (2007)), real-time single molecule coimmunoprecipitation (see, e.g., Lee et al., Nat. Protoc., 8: 2045-2060 (2013)), single molecule electron transfer (see, e.g., Yang et al., Science, 302: 262-266 (2003); and Min et al., Phys. Rev. Lett., 94: 198302 (2005)); single molecule force spectroscopy methods (see, e.g., Capitanio, M. & Pavone, F. S., Biophys. J., 105: 1293-1303 (2013); and Lang et al., Biophys. J., 83: 491-501 (2009)), cell extract pull-down assays (see, e.g., Jain et al., Nature, 473: 484-488, (2011); and Jain et al., Nat. Protoc., 7: 445-452 (2012)), use of molecular motors (see, e.g., Yildiz et al., Science, 300(5628): 2061-2065 (2003)); and single molecule imaging in living cells (see, e.g., Sako et al., Nat. Cell. Biol., 2(3): 168-172 (2000)), nanopore technology (see, e.g., International Patent Application Publication WO 2016/161402), nanowell technology (see, e.g., see, e.g., International Patent Application Publication WO 2016/161400), and single molecule total internal reflection fluorescence (TIRF) microscopy (see, e.g., Reck-Peterson et al., Cold Spring Harb. Protoc., 2010(3):pdb.top73. doi: 10.1101/pdb.top73 (March 2010); and Kukalkar et al., Cold Spring Harb. Protoc., 2016(5):pdb.top077800. doi: 10.1101/pdb.top077800 (May 2016)).

### Device for Analyte Analysis

The methods described herein can be performed using any device suitable for analyte analysis, a variety of which are known in the art and include, for example, peristaltic pump systems (e.g., FISHERBRAND™ Variable-Flow Peristaltic Pumps, ThermoFisher Scientific, Waltham, Mass.; and peristaltic pump systems available from MilliporeSigma, Burlington, Mass.), automated/robotic sample delivery systems (commercially available from e.g., Hamilton Robotics, Reno, Nev.; and ThermoFisher Scientific, Waltham, Mass.), microfluidics devices, droplet based microfluidic devices, digital microfluidics devices (DMF), surface acoustic wave based microfluidic (SAW) devices, or electrowetting on dielectric (EWOD) digital microfluidics devices (see, e.g., Peng et al., Lab Chip, 14(6): 1117-1122 (2014); and Huang et al., PLoS ONE, 10(5): e0124196 (2015)).

In one embodiment, the methods described herein may be performed using a microfluidics microfluidics device, such as a digital microfluidic (DMF) device. Any suitable microfluidics device known in the art can be used to perform the methods described herein. Exemplary microfluidic devices that may be used in the present methods include those described in, for example, International Patent Application Publication Nos. WO 2007/136386, WO 2009/111431, WO 2010/040227, WO 2011/137533, WO 2013/066441, WO 2014/062551, and WO 2014/066704, and U.S. Pat. No. 8,287,808. In certain cases, the device may be a lab-on-chip device, where analyte analysis may be carried out in a droplet of the sample containing or suspected of containing an analyte.

In one embodiment, at least two steps of the method described herein (e.g., 2, 3, or all steps) are carried out in a digital microfluidics device. The terms “digital microfluidics (DMF),” “digital microfluidic module (DMF module),” or “digital microfluidic device (DMF device)” are used interchangeably herein and refer to a module or device that utilizes digital or droplet-based microfluidic techniques to provide for manipulation of discrete and small volumes of liquids in the form of droplets. Complex instructions can be programmed by combining the basic operations of droplet formation, translocation, splitting, and merging.

Digital microfluidics operates on discrete volumes of fluids that can be manipulated by binary electrical signals. By using discrete unit-volume droplets, a microfluidic operation may be defined as a set of repeated basic operations, i.e., moving one unit of fluid over one unit of distance. Droplets may be formed using surface tension properties of the liquid. Actuation of a droplet is based on the presence of electrostatic forces generated by electrodes placed beneath the bottom surface on which the droplet is located. Different types of electrostatic forces can be used to control the shape and motion of the droplets. One technique that can be used to create the foregoing electrostatic forces is based on dielectrophoresis which relies on the difference of electrical permittivities between the droplet and surrounding medium and may utilize high-frequency AC electric fields. Another technique that can be used to create the foregoing electrostatic forces is based on electrowetting, which relies on the dependence of surface tension between a liquid droplet present on a surface and the surface on the electric field applied to the surface.

In another embodiment, the methods described herein may be implemented in conjunction with a surface acoustic wave (SAW) based microfluidic device as a front-end assay processing method. The term “surface acoustic wave (SAW),” as used herein, refers generally to propagating acoustic waves in a direction along a surface. “Travelling surface acoustic waves” (TSAWs) enable coupling of surface acoustic waves into a liquid. In some embodiments, the coupling may be in the form of penetration or leaking of the surface acoustic waves into the liquid. In other embodiments, the surface acoustic waves are Rayleigh waves (see, e.g., Oliner, A. A. (ed), Acoustic Surface Waves. Springer (1978)). Propagation of surface acoustic waves may be conducted in a variety of different ways and by using different materials, including generating an electrical potential by a transducer, such as a series or plurality of electrodes, or by streaming the surface acoustic waves through a liquid.

In some embodiments, the DMF device or the SAW device is fabricated by roll to roll based printed electronics method. Examples of such devices are described in International Patent Application Publication Nos. 2016/161402 and WO 2016/161400.

Many of the devices described above allow for the detection of a single molecule of an analyte of interest. Other devices and systems known in the art that allow for single molecule detection of one or more analytes of interest also can be used in the methods described herein. Such devices and systems include, for example, Quanterix SIMOA™ (Lexington, Mass.) technology, Singulex's single molecule counting (SMC™) technology (Alameda, Calif., see for example, U.S. Pat. No. 9,239,284), and devices described in, for example, U.S. Patent Application Publication Nos. 2017/0153248 and 2018/0017552, or nanopore-based single molecule detection.

### Kits and Cartridges

Also provided herein is a kit for use in performing the above-described methods. The kit may be used with the disclosed device. Instructions included in the kit may be affixed to packaging material or may be included as a package insert. The instructions may be written or printed materials, but are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The kit may include a cartridge that includes a microfluidics module. In some embodiments, the microfluidics module may be integrated in a cartridge. The cartridge may be disposable. The cartridge may include one or more reagents useful for practicing the methods disclosed above. The cartridge may include one or more containers holding the reagents, as one or more separate compositions, or, optionally, as admixture where the compatibility of the reagents will allow. The cartridge may also include other material(s) that may be desirable from a user standpoint, such as buffer(s), a diluent(s), a standard(s) (e.g., calibrators and controls), and/or any other material useful in sample processing, washing, or conducting any other step of the assay. The cartridge may include one or more of the specific binding members described above.

The kit may further comprise reference standards for quantifying the analyte of interest. The reference standards may be employed to establish standard curves for interpolation and/or extrapolation of the analyte of interest concentrations. The kit may include reference standards that vary in terms of concentration level. For example, the kit may include one or more reference standards with either a high concentration level, a medium concentration level, or a low concentration level. In terms of ranges of concentrations for the reference standard, this can be optimized per the assay. Exemplary concentration ranges for the reference standards include but are not limited to, for example: about 10 fog/mL, about 20 fg/mL, about 50 fg/mL, about 75 fg/mL, about 100 fg/mL, about 150 fg/mL, about 200 fg/mL, about 250 fg/mL, about 500 fg/mL, about 750 fg/mL, about 1000 fg/mL, about 10 pg/mL, about 20 pg/mL, about 50 pg/mL, about 75 pg/mL, about 100 pg/mL, about 150 pg/mL, about 200 pg/mL, about 250 pg/mL, about 500 pg/mL, about 750 pg/mL, about 1 ng/mL, about 5 ng/mL, about 10 ng/mL, about 12.5 ng/mL, about 15 ng/mL, about 20 ng/mL, about 25 ng/mL, about 40 ng/mL, about 45 ng/mL, about 50 ng/mL, about 55 ng/mL, about 60 ng/mL, about 75 ng/mL, about 80 ng/mL, about 85 ng/mL, about 90 ng/mL, about 95 ng/mL, about 100 ng/mL, about 125 ng/mL, about 150 ng/mL, about 165 ng/mL, about 175 ng/mL, about 200 ng/mL, about 225 ng/mL, about 250 ng/mL, about 275 ng/mL, about 300 ng/mL, about 400 ng/mL, about 425 ng/mL, about 450 ng/mL, about 465 ng/mL, about 475 ng/mL, about 500 ng/mL, about 525 ng/mL, about 550 ng/mL, about 575 ng/mL, about 600 ng/mL, about 700 ng/mL, about 725 ng/mL, about 750 ng/mL, about 765 ng/mL, about 775 ng/mL, about 800 ng/mL, about 825 ng/mL, about 850 ng/mL, about 875 ng/mL, about 900 ng/mL, about 925 ng/mL, about 950 ng/mL, about 975 ng/mL, about 1000 ng/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 200 μg/mL, about 300 μg/mL, about 400 μg/mL, about 500 μg/mL, about 600 μg/mL, about 700 μg/mL, about 800 μg/mL, about 900 μg/mL, about 1000 μg/mL, about 2000 μg/mL, about 3000 μg/mL, about 4000 μg/mL, about 5000 μg/mL, about 6000 μg/mL, about 7000 μg/mL, about 8000 μg/mL, about 9000 μg/mL, or about 10000 μg/mL.

The kit may include reagents for labeling the specific binding members, reagents for detecting the specific binding members and/or for labeling the analytes, and/or reagents for detecting the analyte. The kit may also include components to elicit cleavage of a tag, such as a cleavage mediated reagent. For example, a cleavage mediate reagent may include a reducing agent, such as dithiothreitol (DTT) or tris(2-carboxyethyl)phosphine) TCEP. The specific binding members, calibrators, and/or controls can be provided in separate containers or pre-dispensed into an appropriate assay format or cartridge.

The kit may also include quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and are useful indicators of the integrity of the kit reagents and the standardization of assays.

The kit may also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit may additionally include one or more other controls. One or more of the components of the kit can be lyophilized, in which case the kit can further comprise reagents suitable for the reconstitution of the lyophilized components. One or more of the components may be in liquid form.

The various components of the kit optionally are provided in suitable containers as necessary. The kit further can include containers for holding or storing a sample (e.g., a container or cartridge for a urine, saliva, plasma, cerebrospinal fluid, or serum sample, or appropriate container for storing, transporting or processing tissue so as to create a tissue aspirate). Where appropriate, the kit optionally can contain reaction vessels, mixing vessels, and other components that facilitate the preparation of reagents or the test sample. The kit can also include one or more sample collection/acquisition instruments for assisting with obtaining a test sample, such as various blood collection/transfer devices (e.g., microsampling devices, micro-needles, or other minimally invasive pain-free blood collection methods; blood collection tube(s); lancets; capillary blood collection tubes; other single fingertip-prick blood collection methods; buccal swabs, nasal/throat swabs; 16-gauge or other size needle, circular blade for punch biopsy (e.g., 1-8 mm, or other appropriate size), surgical knife or laser (e.g., particularly hand-held), syringes, sterile container, or canula, for obtaining, storing or aspirating tissue samples; or the like). The kit can include one or more instruments for assisting with joint aspiration, cone biopsies, punch biopsies, fine-needle aspiration biopsies, image-guided percutaneous needle aspiration biopsy, bronchoaveolar lavage, endoscopic biopsies, and laproscopic biopsies.

The following examples further illustrate the invention but, of course, should not be construed as in any way limiting its scope.

### Example 1

This example describes a method of single molecule counting using total internal reflection fluorescence (TIRF).

Single molecule sample slides were prepared by coating glass slides with drilled holes (50×75 mm, S&S Optical, New Haven, Ind.) and glass coverslips (25×50 mm, Corning, N.Y.) with PEG and PEG/biotin, respectively (MicroSurfaces, Inc., Englewood, N.J.). Rectangular-shaped channels with tapered ends were cut into double-sided tape (9500PC, 3M, Maplewood, Minn.) on a cutting plotter. The tape was sandwiched between the coated slide and coverslip, being careful to prevent air bubbles that might permit leakage, to create the sample wells. There were 6 channels per coverslip, the sample wells were 14 mm long, and each held approximately 5.5, 7 or 8 μL of solution, depending on the width of the channel. Sample solutions were pipetted into the channel through holes in the glass slide located at the ends. Wash steps were performed by pipetting buffer in one end and absorbing the overflow into a tissue at the other end.

All samples were diluted into and washed with HBS-EP buffer (GE Healthcare, Uppsala, Sweden), and all incubation took place at room temperature unless otherwise specified. The detection conjugate used for sensitivity measurements was Alexa Fluor 647-labeled ssDNA (A647-oligo1-bt), with a biotin label at the 3′ end (5′-AlexaF647/CCT TAG AGT ACA AAC GGA ACA CGA GAA/Biot (SEQ ID NO: 1); IDT, Coralville, Iowa). Prior to use, all wells were incubated with 1 μM streptavidin for 20 seconds. A647-oligo1-bt was then incubated for 20-30 minutes at various concentrations ((0, 10, 25, 50, 150, 450 fM, 1, 2, and 4 pM). Each 8-4, well was washed after streptavidin coating and sample incubation, but prior to imaging.

Single molecule total internal reflection fluorescence (SM-TIRF) images were taken on an Olympus IX81 microscope (Center Valley, Pa.) with an attachment for objective-based TIRF. A LIGHTHUB® laser combiner (Omicron, Rodgau, Germany), connected to the microscope via optical fiber, provided four laser wavelengths: 405, 488, 561, and 638 nm. Excitation and emission light passed through a quad filter cube (U-N84000v2; Chroma, Bellows Falls, Vt.), and was focused into the sample with a 100×/1.49 oil immersion TIRF objective. Samples were illuminated with laser powers of approximately 1 mW before the objective, and the images were captured on an iXon Ultra EMCCD camera (Andor, Belfast, UK). SM-TIRF measurements were automated using METAMORPH® Advanced software (Molecular Devices, Sunnyvale, Calif.), and consisted of 40 images per sample well with acquisition times of 150 ms and EM gain of 300. Alexa647 constructs were excited with the 638 nm laser line, and Alexa546 with the 561 nm line. In addition, the Zero Drift autofocus (Olympus Corp., Shinjuku, Tokyo, Japan) was used prior to each image capture to maintain a consistent focus height. Single molecule image data were then analyzed using programs written in IDL 8.5 (Harris Geospatial, Boulder, Colo.). Briefly, the analysis program subtracted a Gaussian background from each image, then located and counted each fluorescent peak above a threshold. Each peak also could be fit to a Gaussian to help eliminate certain types of background. The representative number of single molecule peaks per acquisition was calculated using the median or a resistant mean. Both methods provided nearly identical results. Using the resistant mean rejects frames with outlying peaks/frame values (typically 1-4 frames), and then permits a calculation of the standard deviation of peaks from the remaining 30+ frames.

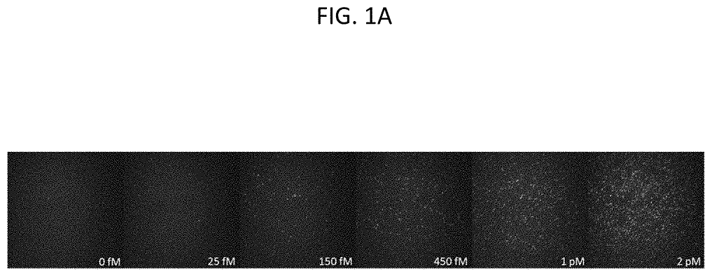

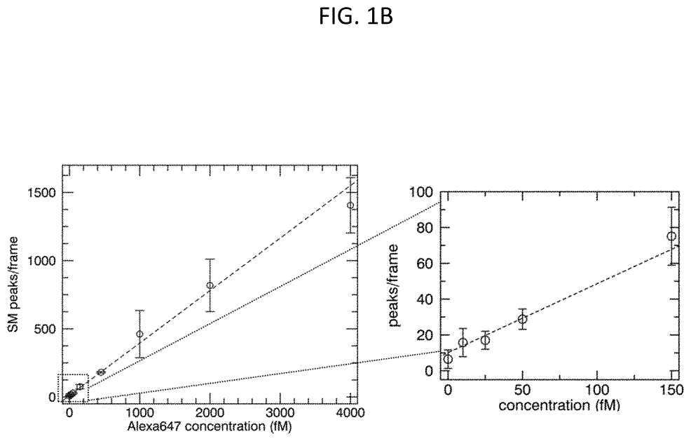

Peaks were shown to correspond to single, immobilized fluorophores. Raw TIRF images are shown in FIG. 1A. A linear dose response was observed from 50 fM to 2 pM, as shown in FIG. 1B. Below 50 fM, it became difficult to separate true sample peaks from the background noise of autofluorescent dust particles and glass impurities (FIG. 1A). Above 2 pM, the high density of peaks made it difficult for the peak finding algorithm to separate closely-spaced molecules, and thus the total count began to saturate. However, higher concentrations could be measured by reverting to a total intensity measurement, rather than digital counting. For a 450 fM sample, assuming that all of the molecules are located on the detection surface, then the calculated upper limit for the average number of molecules per frame was 220. From the data, 181 peaks was the average value; subtracting the background value of 6.5, results in 174.5, or 80% of the maximum expected value.

The results of this example demonstrate the sensitivity of a single molecule TIRF detection system.

### Example 2

This example describes a model system for single molecule detection in an immunoassay.

A model system mimicking a sandwich immunoassay was developed to perform a microparticle-based experiment with a detection label that could be eluted. Specifically, twelve 1-mL samples of a mouse IgG (IgG-oligo2), labeled with DNA oligo2 (5′-TTC TCG TGT TCC GTT TGT ACT CTA AGG TGG ATT TTT TTT TT-amino modifier (SEQ ID NO: 2); IDT, Coralville, Iowa), were prepared by 2×-dilutions from 1024 fM to 1 fM, with a final sample being a buffer-only control. To each sample was added 10 μL of 1%-solid magnetic microparticles (MPs), 5 μm in diameter, which had been directly coated with goat-antimouse antibodies (Abbott Laboratories, Lake Bluff, Ill.). After incubating the samples with rotating at room temperature for 30 minutes, magnetic separation was used to reduce the volume to 200 and the concentrated MP/IgG-oligo2 complexes were transferred to a 96-well plate. Here, on a magnetic particle processor (ThermoFisher Scientific, Waltham, Mass.), the complexes were incubated—mixing at medium speed—with 20 nM A647-oligo1-bt at room temperature for 20 minutes. Subsequently, the MP-sandwich complexes underwent 5 washes in 100 μL ARCHITECT™ wash buffer (Abbott Laboratories, containing PBS), followed by a 10-minute, 85° C. elution step into 50 μL of HBS-EP. This procedure provides a 20-fold reduction in reaction volume from the 1-mL starting sample to the 50-4, eluent.

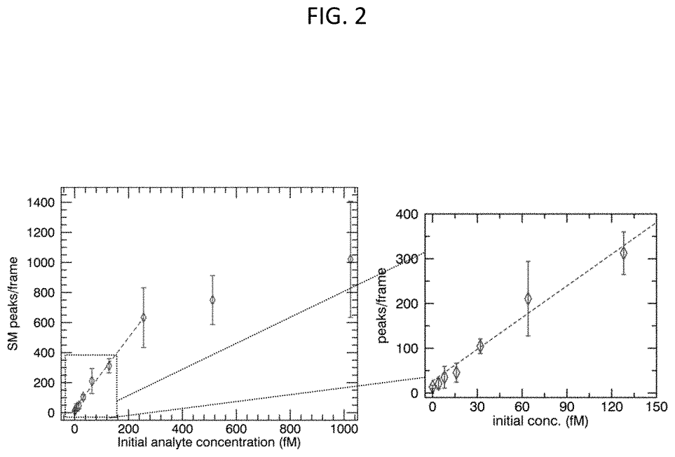

The excess binding sites on the MP (˜5 nM) would have allowed all the analyte from each sample to be bound to the MPs. Using magnetic separation, the volume of each sample was reduced and the concentrated MP complexes were transferred to a 96-well plate. On a microparticle processor, the samples were then incubated with an excess of the detection conjugate, A647-oligo1-bt, and washed 5 times. A 10-minute, 85° C. incubation step was employed to melt the hybridized DNA and elute the A647-oligo1-bt into a small volume (50 μL) of buffer for transfer to the single molecule detection setup. The eluents from each dilution sample were loaded into single molecule wells where the A647-oligo1-bt was anchored to the streptavidin surface via the biotin tags. SM-TIRF images were acquired and processed as described in Example 1. The resulting SM peaks/frame were plotted against the initial analyte concentration values from the sample stocks, as shown in FIG. 2. A linear response was observed with clear sensitivity down to the original sample concentration range of approximately 20-30 fM. Due to the 20-fold reduction in volume from the starting sample to the eluent and the roughly 50% capture efficiency of the microparticles, the detection label concentrations actually measured were about 10 times higher than the starting values. Therefore, the saturation that occurred in the two highest concentrations fell in the >2 pM range, consistent with previous observations.

### Example 3

This example demonstrates a method for concentrating an analyte present in a biological sample through sample reloading.

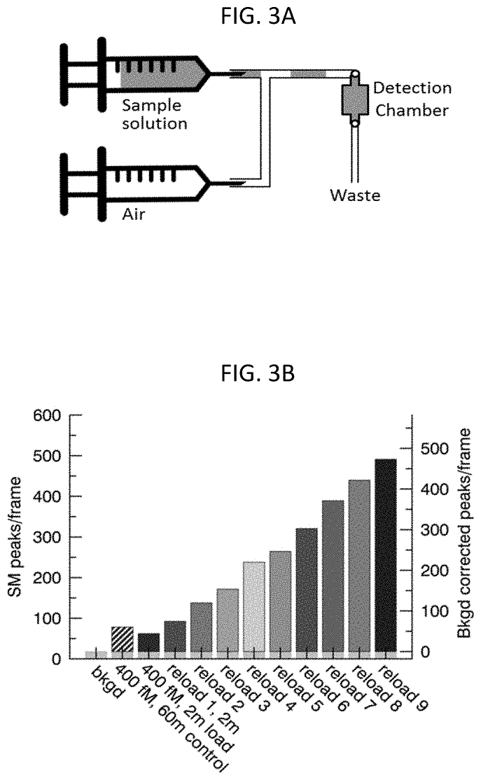

Single molecule detection methods typically require only a small sample volume. Taking advantage of small sample volume requirements, a strategy of cyclically reloading fresh aliquots of the same sample stock was developed to concentrate the sample prior to detection and enhance assay sensitivity. By incubating each aliquot for only 1-2 minutes and then replacing it with fresh stock, a sample may be concentrated onto the surface of a slide. For example, an aliquot of a stock of 400 fM A546-oligo1-bt was loaded into an SM well, a measurement was performed, and then the aliquot was replaced with a fresh aliquot of the stock 10 times, measuring after each 2-minute incubation. The previous aliquot was cleared out of the well by pumping air through the well in between reloads, as shown in the schematic of FIG. 3A. The surface of the well was not allowed to dry, but rather an air gap approximately the volume of air necessary to fill the sample well was transiently introduced into the well, which broke up the continuous flow of liquid. In contrast, loading a new sample aliquot directly into the well did not push out the previous aliquot, which is likely due to the fact that there was not consistent, plug-like, laminar flow such that the fresh stock partially mixed with—or even fully passed over—a stationary surface layer of the exhausted aliquot solution. With an air gap between reloads, however, remarkably consistent concentration results were observed, as shown in FIG. 3B.

Due to the strength of streptavidin-biotin interactions and the 150-μm well height used for these experiments, most of the available targets had diffused to, and were captured within, the selected 2-minute incubation period. However, given a weaker capture interaction or a taller sample compartment, it may also be possible to gain signal from sample recycling, i.e., removing the sample, replacing it with air, and immediately reloading the same aliquot. In the above-described experiments, each reloading step increased the observed number of SM peaks by an average of 43 peaks, which is 70% of the number of peaks captured from the fully saturated, 1-hour incubation. The variation in the numbers of peaks/reload was less than 10%, thus, after nine reloads on top of the initial load, a 10-fold increase in the number of background-corrected peaks was observed.

The above results demonstrate that the specific number of surface captures in each reloading step depends on both the selected incubation time and the surface binding kinetics.

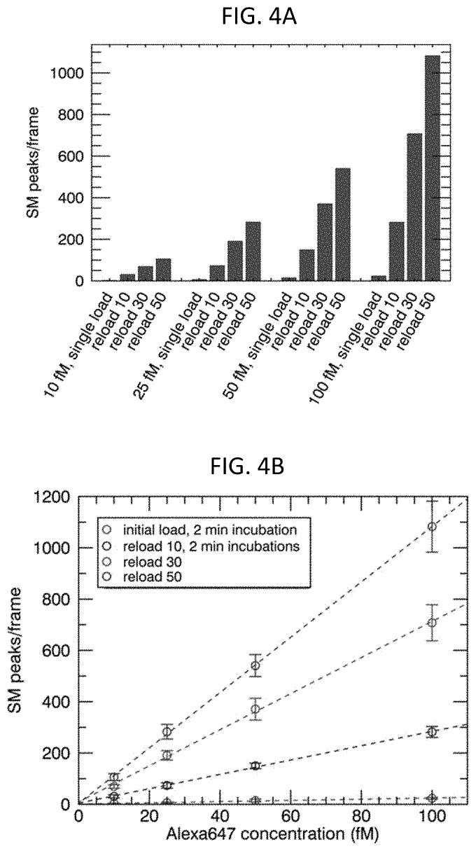

To determine whether the sample reloading method may be employed in a dose-response style assay, the same reloading steps were performed on four concentrations (10, 25, 50, and 100 fM) of A647-oligo1-bt using 2-minute incubations, and the results after the initial load, the 10th reload, the 30th reload, and the 50th reload were measured. The results of this experiment are shown in FIGS. 4A and 4B, which shows that the linear relationship as a function of concentration was maintained throughout the reloading. To properly determine the fold-enhancement of reloading, it was necessary to subtract the background of the empty well. The initial reloading experiments were performed with Alexa546-labeled conjugates. However, in the absence of sample, the green channel typically exhibited 10-20 fluorescent peaks, which were believed to be impurities and/or dust in the coverslip glass. While this is useful for demonstrating how reloading can boost the target signal out of this type of background, Alexa647 was selected for all other experiments due to the lower background (5-10 peaks) observed in the red channel. Once the surface background correction was applied, however, n reloads concentrated all starting sample concentrations by very nearly n-fold.

The results of this example demonstrate that the disclosed sample reloading method is an effective concentration method to enhance the detection of unknown, low concentration diagnostic samples.

### Example 4

This example describes an assay for single molecule detection of the HIV p24 antigen.

A full sandwich immunoassay was conducted to detect p24, an HIV capsid protein commonly detected in diagnostic assays for HIV. Specifically, eight TIRF slide wells were incubated with 1 μM streptavidin for 20 seconds, then washed with 2×100 μL of HBS-EP. Eight 200-4, samples of p24 antigen (Abbott Laboratories, Lake Bluff, Ill.) were prepared by 2-fold dilutions with a buffer control (0, 40, 80, 160, 320, 640 fM, 1.28 pM, & 2.56 pM). The samples were transferred to a 96-well plate and 50 μL of 0.1% solids, anti-p24 antibody-coated MPs were added to each sample (final volume, 250 On a KINGFISHER™ magnetic microparticle processor (ThermoFisher Scientific, Waltham, Mass.), the samples were mixed and incubated for 18 minutes at room temperature. This was followed by a wash with ARCHITECT™ (Abbott Diagnostics, Lake Forest, Ill.) wash buffer and a second 18-minute incubation with the detection conjugate. The detection conjugate consisted of 0.5 nM of an Abbott anti-p24 Fab, labeled with oligo2, and preassembled (2 hours, 37° C.) with 2 nM of A647-oligo1-bt. The completed MP-bound immunosandwiches were passed through four more washes and then the A647-oligo1-bt was eluted off by a 10-minute, 85° C. elution step into 250 μL of HBS-EP. The eluent was loaded into SM wells, incubated for two minutes, washed with HBS-EP, and measured with SM-TIRF. Fresh aliquots of the eluent solutions were then added every two minutes, 9 more times, for a total of 10 aliquots of sample captured on the surface of each well.

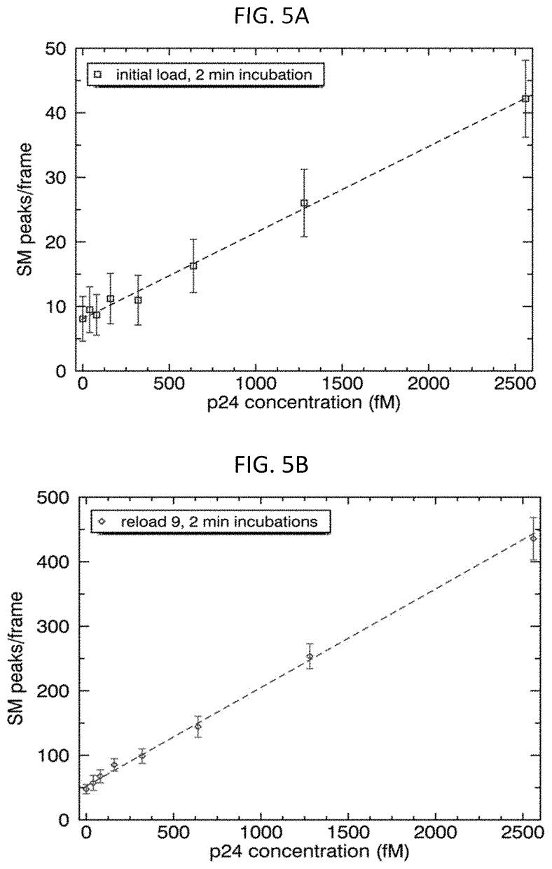

The results of the immunoassay following first elution of the A647-oligo1-bt from the microparticle-bound SM-TIRF are shown in FIG. 5A, which demonstrates a linear response, but the numbers of peaks were low and the error large. After reloading aliquots from each eluted sample 9 more times, for a total of ten 2-minute surface captures, remeasuring the SM wells demonstrated a roughly 10-fold increase in raw signal and a 3-fold reduction in relative error, as show in FIG. 5B.

The results of this example demonstrate that the disclosed repeat sampling method can be applied to an immunoassay for detection of an HIV antigen.

### Example 5

This example demonstrates that the disclosed sample reloading approach enhances immunoassay sensitivity when using digital microfluidics (DMF).

A model immunoassay using a 3-step format consisting of antigen capture, biotinylated conjugate binding, and enzyme labeling with a streptavidin-enzyme conjugate was tested. The use of digital microfluidics (DMF) allows the manipulation of small sample volumes (<2 μl), which has an advantage of increasing the capture efficiency of antibody-antigen binding when solid-phase binding is used. The modeling experiment described below was performed to demonstrate the advantage of DMF-based immunoassays using small volumes to increase assay sensitivity.

The modeling algorithm was derived from L. Chang, et al., J. Immun. Methods, 378: 102-115 (2012) using the following equation to determine the overall rate of formation of antibody-ligand complexes:

$$\frac{\partial [ AbL ]}{\partial t} = {k}_{on} ( [ {Ab}_{total} ] - [ AbL ] ) ( [ {L}_{total} ] - [ AbL ] ) - {k}_{off} [ AbL ] .$$

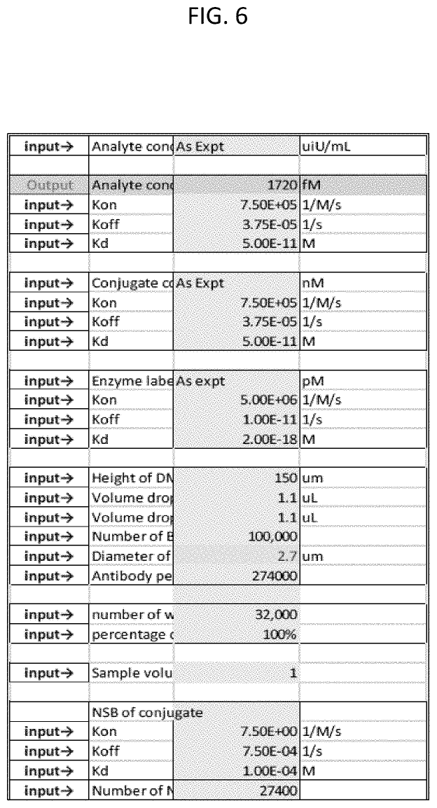

The rate of complex formation may be plotted in real-time using kₒₙ and k_{off} rates for the specific antibody-antigen pair. For antigen capture, antibodies are assumed to be covalently attached to the surface of magnetic microparticles for solid-phase capture of antigen. Input parameters for the experiment are shown in FIG. 6, and experimental conditions are shown below in Tables 1 and 2.

TABLE 1

Liquid volume in all steps = 1.1 μL
Number of beads = about 100 000
Conjugate concentration = 10 nM
SBG concentration = 150 pM
Incubation time TSH = 5 min
Incubation time conjugate = 5 min
Incubation time SBG = 5 min

TABLE 2

| repetitions | sample vol, μl | capture time, min. |
|---|---|---|
| 1 | 1.1 | 5 |
| 1 | 5.5 | 5 |
| 5 | 1.1 | 1 each |

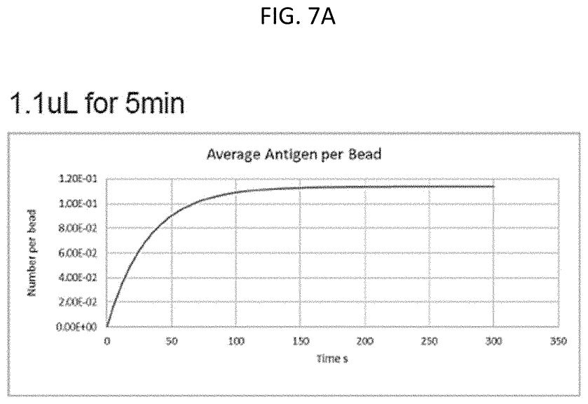

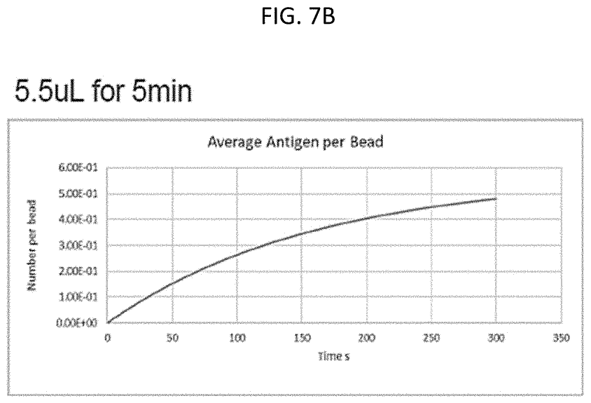

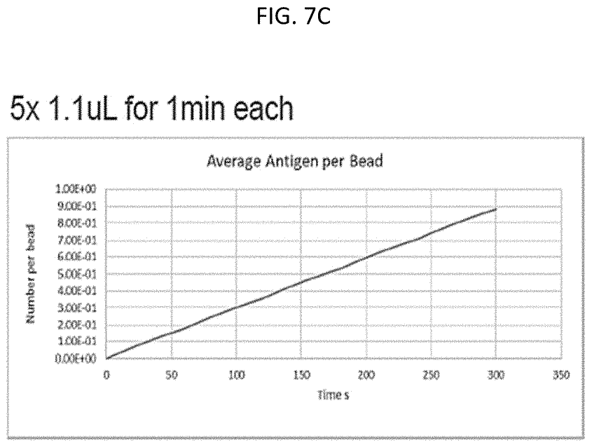

Real-time antigen binding curves for the three different conditions shown in Table 2 during the first five minutes of incubation are shown in FIGS. 7A-7C.

Labeling of captured antigen on microparticles was modeled using 10 nM biotinylated conjugate antibody for 5 minutes, followed by a 5-minute enzyme labeling step using 150 pM streptavidin-β-galactosidase (SBG). Final average enzymes per bead (AEB) were calculated and are shown below in Table 3.

TABLE 3

| Sample Conditions | AEB |
|---|---|
| 1 × 1.1 μl for 5 min | 0.020 |
| 5.5 μl for 5 min | 0.086 |
| 5 × 1.1 μl for 1 min | 0.158 |

These results show that the sample re-loading protocol produces a final AEB signal that is approximately two times higher than a single loading protocol (0.158 AEB vs. 0.086 AEB). Using the same number of beads, the smaller volume (1.1 μl) on the DMF device allowed for a higher bead:volume ratio. This raises the effective capture antibody concentration in the capture step, thereby increasing the rate at which antigen binds to the antibody-bound microparticles. In the binding curve example for 1.1 μl with 5-minute incubation, most of the antigen is bound within the first minute of incubation.

Re-loading the sample multiple times using shorter incubation times increased the amount of antigen captured as compared to a higher sample volume with a longer incubation time, because maximal binding takes longer in the larger volume due to the lower bead:volume ratio.

### Example 6

A digital assay for detecting thyroid stimulating hormone (TSH) was run on a 2″×3″ digital microfluidic (DMF) chip, using a microwell array (32,000 wells) for digital detection. A droplet (1.1 μl) containing TSH (buffer=SuperBlock, 1.5% BSA, 0.05% Tween-20, 0.1% F68) was moved to a microparticle pellet containing approximately 100K beads labeled with TSH capture antibody (M4, Fitzgerald). The beads were mixed for 5 minutes followed by pelleting. The pellet was suspended in wash buffer (SuperBlock, 1.5% BSA, 0.05% Tween-20, 0.1% F68) and washed by mixing for 2 minutes followed by pelleting. The washed pellet was suspended in 1.1 μl buffer containing 1 nM biotinylated conjugate antibody (ME-130, Abcam) and mixed for 5 minutes followed by pelleting. The pellet was suspended in wash buffer (SuperBlock, 1.5% BSA, 0.05% Tween-20, 0.1% F68) and washed by mixing for 2 minutes followed by pelleting. Approximately 1.1 μl of 150 pM streptavidin-β-galactosidase was added to the pellet. The beads were mixed for 5 minutes followed by pelleting. The pellet was suspended in wash buffer (SuperBlock, 1.5% BSA, 0.05% Tween-20, 0.1% F68) and washed by mixing for 2 minutes followed by pelleting. The beads were prepared for seeding by adding 1.1 μl seeding buffer (1×PBS, 0.05% Tween-20) and mixing for 2 minutes. The mixture was moved to the microwell array, followed by addition of 1.1 μl 152 μM resorufin-D-galactopyranoside (RGP) enzymatic substrate (1×PBS, 0.05% Tween-20) at 35° C. The temperature was decreased to 27.5° C. before seeding with circular motion of the droplet over the array. The RGP droplet was removed, the temperature was reduced to ˜8° C., followed by oil sealing with Krytox 1525 oil. Dark field and fluorescence imaging was taken after 1 hour of enzymatic turnover.

For 3λ re-loading, the same protocol was used, except the initial sample loading was repeated three times before conjugate addition. Average enzymes per bead (AEB) were calculated from % active beads (fₒₙ) by using the following conversion: AEB=−ln[1−fₒₙ], and the results are shown in Table 4.

TABLE 4

| [TSH], μIU/ml | AEB, raw | AEB, bkgd sub |
|---|---|---|
| 0 | 0.285 | 0 |
| 1X 0.05 | 0.327 | 0.042 |
| 3X 0.05 | 0.380 | 0.095 |

A 3× re-loading of 0.05 μIU/ml resulted in a sensitivity increase of approximately 2.3-fold.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

For reasons of completeness, various aspects of the invention are set out in the following numbered clauses:

Clause 1. A method for detecting an analyte present in a biological sample, which method comprises:

(a) providing a volume of a biological sample suspected of containing an analyte;

(b) contacting a solid support with a first aliquot of the volume of the biological sample, wherein the solid support comprises immobilized thereto a first specific binding member that specifically binds to the analyte;

(c) removing the first aliquot from the solid support and contacting the solid support with a second aliquot of the volume of the biological sample;

(d) repeating steps (b) and (c) 5 to 30 times, wherein a solid support/first specific binding member/analyte complex is formed;

(e) contacting the solid support/first specific binding member/analyte complex with a second specific binding member that specifically binds to the analyte and comprises a detectable label attached thereto, wherein a solid support/first specific binding member/analyte/second specific binding member complex is formed;

(f) removing any second specific binding member not bound to the analyte; and

(g) detecting the analyte by assessing a signal produced by the detectable label.

Clause 2. A method for detecting an analyte present in a biological sample, which method comprises:

(a) providing a volume of a biological sample suspected of containing an analyte;

(b) contacting a solid support with a volume of the biological sample, wherein the solid support comprises immobilized thereto a first specific binding member that specifically binds to the analyte;

(c) contacting the solid support/first specific binding member/analyte complex with a second specific binding member that specifically binds to the analyte and comprises a detachable detectable label attached thereto, wherein a solid support/first specific binding member/analyte/second specific binding member complex is formed;

(d) separating and eluting the detectable label from complex bound to the solid support;

(e) transferring an aliquot of detectable label to a second solid support comprising a third specific binding member that specifically binds the detectable label;

(f) removing the first aliquot from the solid support and contacting the solid support with a second aliquot of the eluted detectable label;

(g) repeating steps (e) and (f) 5 to 30 times, wherein a solid support/third specific binding member/detectable label complex is formed;

(h) removing any detectable label not bound to the solid support; and

(i) quantifying the analyte by assessing a signal produced by the detectable label.

Clause 3. The method of clauses 1 or 2, wherein the volume of the biological sample is about 10 μl to about 50 μl.

Clause 4. The method of clauses 1 to 3, wherein the first and second aliquots comprise about 1 μl to about 2 μl of the solution volume.

Clause 5. The method of clause 4, wherein the first and second aliquots comprise about 1 μl of the solution volume.

Clause 6. The method of any one of clauses 1 to 5, wherein the analyte is a protein, a glycoprotein, a peptide, an oligonucleotide, a polynucleotide, an antibody, an antigen, a hapten, a hormone, a drug, an enzyme, a lipid, a carbohydrate, a ligand, or a receptor.

Clause 7. The method of any one of clauses 1 to 6, wherein the first and/or second binding member is an antibody, a receptor, a peptide, or a nucleic acid sequence.

Clause 8. The method of any one of clauses 1 to 7, wherein the solid support is a particle, a microparticle, a bead, an electrode, a slide, or a multiwell plate.

Clause 9. The method of clause 8, wherein the first solid support is a microparticle and the second solid support is a slide.

Clause 10. The method of clause 9, wherein the microparticle is magnetic.

Clause 11. The method of any one of clauses 1 to 10, wherein the biological sample is blood, serum, plasma, urine, saliva, sweat, sputum, or semen.

Clause 12. The method of any one of clauses 1 toll, wherein the detectable label comprises a chromagen, a fluorescent compound, an enzyme, a chemiluminescent compound, or a radioactive compound.

Clause 13. The method of any one of clauses 1 to 12, wherein at least steps (1b) and (1c) or (2e) and (2f) are carried out in a microfluidics device, a droplet based microfluidic device, a digital microfluidics device (DMF), or a surface acoustic wave based microfluidic device (SAW).

Clause 14. The method of any one of clauses 1 to 13, wherein a signal produced by the detectable label is assessed using an immunoassay.

Clause 15. The method of clause 14, wherein the immunoassay is a sandwich immunoassay, an enzyme immunoassay (EIA), an enzyme-linked immunosorbent assay (ELISA), a competitive inhibition immunoassay, an enzyme multiplied immunoassay technique (EMIT), a competitive binding assay, a bioluminescence resonance energy transfer (BRET), a one-step antibody detection assay, or a homogeneous chemiluminescent assay.

Clause 16. The method of any one of clauses 1 to 15, which detects a single molecule of the analyte.

1. A method for detecting an analyte present in a biological sample, which method comprises:
(a) providing a volume of a biological sample suspected of containing an analyte;
(b) contacting a solid support with a first aliquot of the volume of the biological sample, wherein the solid support comprises immobilized thereto a first specific binding member that specifically binds to the analyte;
(c) removing the first aliquot from the solid support and contacting the solid support with a second aliquot of the volume of the biological sample;
(d) repeating steps (b) and (c) 5 to 30 times, wherein a solid support/first specific binding member/analyte complex is formed;
(e) contacting the solid support/first specific binding member/analyte complex with a second specific binding member that specifically binds to the analyte and comprises a detectable label attached thereto, wherein a solid support/first specific binding member/analyte/second specific binding member complex is formed;
(f) removing any second specific binding member not bound to the analyte; and
(g) detecting the analyte by assessing a signal produced by the detectable label.

2. A method for detecting an analyte present in a biological sample, which method comprises:
(a) providing a volume of a biological sample suspected of containing an analyte;
(b) contacting a solid support with a volume of the biological sample, wherein the solid support comprises immobilized thereto a first specific binding member that specifically binds to the analyte;
(c) contacting the solid support/first specific binding member/analyte complex with a second specific binding member that specifically binds to the analyte and comprises a detachable detectable label attached thereto, wherein a solid support/first specific binding member/analyte/second specific binding member complex is formed;
(d) separating and eluting the detectable label from complex bound to the solid support;
(e) transferring an aliquot of detectable label to a second solid support comprising a third specific binding member that specifically binds the detectable label;
(f) removing the first aliquot from the solid support and contacting the solid support with a second aliquot of the eluted detectable label;
(g) repeating steps (e) and (f) 5 to 30 times, wherein a solid support/third specific binding member/detectable label complex is formed;
(h) removing any detectable label not bound to the solid support; and
(i) quantifying the analyte by assessing a signal produced by the detectable label.

3. The method of claim 1, wherein the volume of the biological sample is about 10 μl to about 50 μl.

4. The method of claim 1, wherein the first and second aliquots comprise about 1 μl to about 2 μl of the solution volume.

5. The method of claim 4, wherein the first and second aliquots comprise about 1 μl of the solution volume.

6. The method of claim 1, wherein the analyte is a protein, a glycoprotein, a peptide, an oligonucleotide, a polynucleotide, an antibody, an antigen, a hapten, a hormone, a drug, an enzyme, a lipid, a carbohydrate, a ligand, or a receptor.

7. The method of claim 1, wherein the first and/or second binding member is an antibody, a receptor, a peptide, or a nucleic acid sequence.

8. The method of claim 1, wherein the solid support is a particle, a microparticle, a bead, an electrode, a slide, or a multiwell plate.

9. The method of claim 8, wherein the first solid support is a microparticle and the second solid support is a slide.

10. The method of claim 9, wherein the microparticle is magnetic.

11. The method of claim 1, wherein the biological sample is blood, serum, plasma, urine, saliva, sweat, sputum, or semen.

12. The method of claim 1, wherein the detectable label comprises a chromagen, a fluorescent compound, an enzyme, a chemiluminescent compound, a nucleic acid molecule, or a radioactive compound.

13. The method of claim 1, wherein at least steps (1b) and (1c) are carried out in a microfluidics device, a droplet based microfluidic device, a digital microfluidics device (DMF), or a surface acoustic wave based microfluidic device (SAW).

14. The method of claim 1, wherein a signal produced by the detectable label is assessed using an immunoassay.

15. The method of claim 14, wherein the immunoassay is a sandwich immunoassay, an enzyme immunoassay (EIA), an enzyme-linked immunosorbent assay (ELISA), a competitive inhibition immunoassay, an enzyme multiplied immunoassay technique (EMIT), a competitive binding assay, a bioluminescence resonance energy transfer (BRET), a one-step antibody detection assay, or a homogeneous chemiluminescent assay.

16. The method of claim 1, which detects a single molecule of the analyte.
